(19) United States
(12) Patent Application Publication
(10) Pub. No.: US 2006/0090214 A1
(43) Pub. Date: Apr. 27, 2006

(54) Method of treatment and an animal model useful for same

(76) Inventors: Szuanne Cory, North Melbourne (AU); Jerry Adams, North Melbourne (AU); Cris Print, North Melbourne (AU); Leonie Gibson, Moonee Ponds (AU); Frank Koentgen, Park Orchards (AU)

Correspondence Address: Scully, Scott, Murphy & Presser, 400 Garden City Plaza, Garden City, NY, 11530, US

(21) Appl. No.: 11/301,951

(22) Filed: Dec. 13, 2005

Related U.S. Application Data
(63) Continuation of application No. 09/508,745, filed on Jul. 12, 2000.

(30) Foreign Application Priority Data
Sep. 16, 1997 (AU) PO 9228
Sep. 16, 1998 (WO) PCT/AU98/00764

(51) Int. Cl. A01K 67/027

(52) U.S. Cl. 800/18; 800/19

(57) ABSTRACT

The present invention relates generally to a method of treatment and to an animal model for the identification of molecules and genetic sequences useful in method of treatment including inducing or reducing fertility of male animals. More particularly, the present invention contemplates a method for the treatment of infertility or a method of reducing fertility and even more particularly a method for modulating spermatogenesis in an animal or avian species. There is also provided an animal model comprising a mutation in at least one allele of bcl-w or in a gene associated with bcl-w. Such animals fail to undergo productive spermatogenesis and can be used to screen for therapeutic molecules including genetic sequences capable of inducing, enhancing or otherwise facilitating spermatogenesis in said animals as well as a model for molecules and genetic sequences which can induce infertility.

### CROSS REFERENCE TO RELATED APPLICATIONS

This application is a continuation of U.S. application Ser. No. 09/508,745, filed Jul. 12, 2000 as a 371 application based on PCT/AU98/00764 having an international filing date of Sep. 16, 1998.

### FIELD OF THE INVENTION

The present invention relates generally to a method of treatment and to an animal model for the identification of molecules and genetic sequences useful in a method of treatment including inducing or reducing the fertility of male animals. More particularly, the present invention contemplates a method for the treatment of infertility or a method of reducing fertility and even more particularly a method for modulating spermatogenesis in an animal or avian species. There is also provided an animal model comprising a mutation in at least one allele of bcl-w or in a gene associated with bcl-w. Such animals fail to undergo productive spermatogenesis and can be used to screen for therapeutic molecules including genetic sequences capable of inducing, enhancing or otherwise facilitating spermatogenesis in said animals as well as a model for molecules and genetic sequences which can induce infertility.

### BACKGROUND OF THE INVENTION

Bibliographic details of the publications numerically referred to in this specification are collected at the end of the description.

Considerable effort has and continues to be expended on therapeutic protocols for the treatment of genetically based disorders. To facilitate the rationale design of such therapeutic protocols, scientists first need to understand and elucidate the biochemical and genetic intricacies of intracellular pathways and physiological processes. Several key regulators have been identified which have involvement in intracellular pathways and physiological processes. A particularly important group of proteins is the Bcl-2 family of proteins.

Bcl-2 is a 26 kDa cytoplasmic protein encoded by the bcl-2 gene translocated to the IGH locus in human follicular lymphoma and is regarded as the prototypic mediator of cell survival (1). The Bcl-2 proteins have a role in controlling cellular apoptosis. Apoptosis is a morphologically distinctive and genetically programmed process of cell death (2) and plays an important role in embryogenesis, tissue homeostasis and the immune system.

Disrupted regulation of apoptosis is strongly implicated in cancer and in autoimmune and degenerative diseases. Key regulators include proteins of the Bcl-2 family (reviewed in 3-5), some of which (eg Bcl-2, Bcl-x_{L}, Mcl-1 and A1) promote cell survival while others (eg Bax, Bak) act as antagonists. Because members of these opposing factions can associate and seemingly titrate one another's function, their relative abundance in a particular cell type may determine its threshold for apoptosis (6). The competitive action of the pro- and anti-survival Bcl-2-related proteins regulates the activation of the proteases (caspases) that dismantle the cell, but how they do so remains uncertain (3-5). The pro-survival proteins may, however, associate with caspase-activating adaptors such as Ced-4 and Apaf-1 and prevent their activity (7-8) and/or prevent the release of pro-apoptotic proteins from mitochondria (9, 10, 11).

The pro-survival family members are expressed in diverse tissues in distinct but overlapping patterns. While their biochemical actions are difficult to distinguish, gene inactivation studies suggest that each may have critical roles in particular tissues. Mice which lack Bcl-2 develop normally, but later display marked lymphocytopenia, polycystic kidney disease, hypopigmented hair, motoneuron degeneration and disordered growth of intestinal villi and long bones (12-17). In contrast, mice which lack Bcl-x_{L} die in utero due to massive apoptosis of both hematopoietic and neuronal cells (18).

Bcl-w is a pro-survival protein identified by the present inventors (19; International Patent Application No. PCT/AU97/00199, filed 27 Mar., 1997 and incorporated herein by reference). Enforced expression of bcl-w, like bcl-2, renders myeloid and lymphoid cell lines refractory to apoptosis induced by cytokine deprivation or irradiation, but is relatively ineffective against apoptosis induced by engagement of the CD95 (Fas) ‘death’ receptor. Transcripts of bcl-w are present at moderate levels in brain, colon and salivary gland, and at low levels in testis, liver, heart, stomach, skeletal muscle and placenta, as well as in most myeloid cell lines but few lymphoid lines (19).

In work leading up to the present invention and in order to identify in which tissues Bcl-w plays an essential role, the inventors undertook bcl-w gene disruption studies in mice. It has now been surprisingly determined that mice deficient for bcl-w and/or a gene associated with bcl-w fail to undergo productive spermatogenesis and are infertile without showing any other major abnormality. In contrast, Bcl-w is apparently dispensable in other tissues. The mice provide, therefore, a useful model for studying infertility in animal and avian species.

### SUMMARY OF THE INVENTION

Sequence Identity Numbers (SEQ ID NOs.) for the nucleotide and amino acid sequences referred to in the specification are defined following the bibliography.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

One aspect of the present invention is directed to a modified animal or avian species exhibiting reduced levels of a Bcl-w protein and/or a protein associated with Bcl-w or a derivative or homologue thereof, wherein said animal or avian species has an incapacity or a reduced capacity to induce or facilitate spermatogenesis.

Another aspect of the present invention provides a modified animal or avian species exhibiting reduced levels of a Bcl-w protein having an amino acid sequence substantially as set forth in SEQ ID NO:2 or SEQ ID NO:4 or a Bcl-w protein encoded by a nucleotide sequence substantially set forth in SEQ ID NO:1 or SEQ ID NO:3 or a nucleotide sequence capable of hybridising to SEQ ID NO:1 or 3 or 5 or 7 under low stringency conditions at 42° C. wherein said animal or avian species has an incapacity or a reduced capacity to induce or facilitate spermatogenesis.

Yet another aspect of the present invention provides a modified animal exhibiting reduced levels of Bcl-w or a derivative or homologue thereof and/or of a protein associated with Bcl-w wherein said Bcl-w or its derivative or homologue comprises an amino acid sequence substantially as set forth in SEQ ID NO:2 or SEQ ID NO:4 or an amino acid sequence having at least about 47% similarity to the amino acid sequence of SEQ ID NO:2 or SEQ ID NO:4 and wherein said modified animal has a incapacity or a reduced capacity to induce or facilitate productive spermatogenesis.

Still yet another aspect of the present invention contemplates a modified animal exhibiting reduced levels of Bcl-w or a derivative or homologue thereof and/or of a protein associated with Bcl-w wherein said Bcl-w or its derivative or homologue is encoded by a nucleotide sequence substantially as set forth in SEQ ID NO:1 or SEQ ID NO:3 or a nucleotide sequence having at least 47% similarity thereto and/or which can hybridise to SEQ ID NO:1 or SEQ ID NO:3 under low stringency conditions at 42° C.

Another aspect of the present invention is directed to a modified animal exhibiting an incapacity or a reduced capacity to induce or facilitate productive spermatogenesis said modification comprising the administration to said animal of an antagonistic effective amount of a molecule capable directly or indirectly of antagonising Bcl-w protein activity or the ability of a derivative or homologue of Bcl-w.

Yet another aspect of the present invention provides a composition capable of inducing infertility or reducing fertility in an animal, said composition comprising a direct or indirect antagonist of a Bcl-w protein.

Still yet another aspect of the present invention relates to a genetically modified animal comprising a mutation in one or more alleles of a gene encoding a Bcl-w protein and/or of a gene encoding a molecule associated with Bcl-w protein.

Even yet another aspect there is provided a genetically modified animal comprising a mutation in one or more alleles of a gene comprising a sequence of nucleotides substantially as set forth in SEQ ID NO:1 or SEQ ID NO:3 or a nucleotide sequence having at least about 47% similarity thereto and/or a sequence which is capable of hybridising to SEQ ID NO:1 or SEQ ID NO:3 under low stringency conditions at 42° C.

Even still another aspect of the present invention contemplates a method of producing a genetically modified animal substantially incapable of producing Bcl-w, said method comprising introducing a genetic sequence into embryonic stem (ES) cells, which genetic sequence targets the bcl-w gene or a gene associate with bcl-w and introducing said ES cells into blastocysts to produce chimeric mice.

Another aspect of the present invention contemplates transgenic animals such as mice containing a genetic sequence operably linked to a testis-specific promoter, which genetic sequence is capable of disrupting the bcl-w gene or bcl-w gene expression or expression of a gene associated with bcl-w in the testis.

Yet another aspect of the present invention is directed to a modified animal comprising a mutation in a gene corresponding to bcl-w or a derivative or homologue thereof or in a gene associated with bcl-w wherein an adult male of said animal exhibits the following characteristics:
(i) is substantially infertile;
(ii) possesses disorganised seminiferous tubules;
(iii) exhibits heterogenous degeneration of germ cell types; and
(iv) possesses no other major abnormalities as determined by histological examination.

Still yet a further aspect of the present invention contemplates an animal model for studying other degenerative disorders such as but not limited to neurodegenerative disorders.

### BRIEF DESCRIPTION OF THE FIGURES

FIGS. 1A-1F show the disruption of the bcl-w gene. (A) The targeting vector pbcl-wlox neo^{r} tk. Shaded bars represent regions derived from the bcl-w gene; tk, a thymidine kinase expression cassette; neo^{r}, a PGK-neo^{r} expression cassette; and diamonds, loxP sequences. (B) The wt bcl-w locus. Boxes represent exons (solid, coding region; open, untranslated region). E, Eco RI sites; sizes of Eco RI fragments are in kb. The bcl-w genomic DNA probes used for Southern blot analyses are labeled a and b, while the bcl-w cDNA sequences used as riboprobes are indicated by c and d. (C) Homologous recombination replaces the first 413 bp of the bcl-w coding region with a PGK-neo^{r} expression cassette bounded by loxP sites. (D) Cre-mediated recombination deletes the PGK-neo^{r} sequence, leaving only 127 bp of exogenous sequence, including a single loxP site. (E) Southern blot of genomic DNA from wt (+/+), heterozygous (+/−) and homozygous mutant (−/−) bcl-w mice (line 228), hybridized with bcl-w cDNA probe a. (F) Southern blot of genomic DNA from heterozygous mice (line 228) before (+/−) and after (+/Δ) the action of Cre recombinase, hybridized with bcl-w probe b.

FIGS. 2A-2C are photographic representations showing expression of the bcl-w gene. (A) Northern blot of total RNA (10 μg) extracted from the testes of 4-wk old wt (+/+) and bcl-w^{Δ/Δ} mice (Δ/Δ), hybridized to a probe containing the first 1.2 kb of the bcl-w cDNA (upper panel); glyceraldehydes phosphate dehydrogenase mRNA served as a control (gapdh, lower panel). (B) Western blot analysis of protein lysates from the brain, testis and pancreas of wt and bcl-w^{Δ/Δ} mice, using polyclonal anti-Bcl-w antibody. The 21-kDa Bcl-w protein is indicated. (C) Western blots of protein lysates from testis cell lines, with the same antibody. GC-1 is a germ cell line derived from type B spermatogonia, TM4 a Sertoli cell line and TM3 a Leydig cell line; all were obtained from the American Type Culture Collection.

FIG. 3 is a graphical representation showing reduced numbers of various cell types within the seminiferous tubules of bcl-w^{Δ/Δ} mice. Frequencies of the indicated cell types was determined by the optical disector method for seven 6 wk-old wt mice and eight 6 wk-old bcl-w^{Δ/Δ} mice. The percentage of the wt cell numbers remaining in the testes of bcl-w^{Δ/Δ} mice is indicated. Error bars denote 2 standard errors of the means (SEM).

FIGS. 4A-4B are graphical representations showing degeneration of testis in bcl-w^{Δ/Δ} mice. (A) Mean mass of testes (3 mice per group). (B) TUNEL-labelled nuclei per tubule, counted at 2, 4, 8 and 14 wk (3 mice per group). Error bars denote 2 SEM.

FIG. 5 is a diagrammatic representation of the consequences of Bcl-w loss in the testis. The percentages of the Sertoli cells and the different types of germ cells remaining in bcl-w^{Δ/Δ} mice are indicated. The expression pattern of the gene is indicated schematically; the broken line indicates that the extent of expression in late stages of germ cell development remains to be clarified.

The following abbreviations are used in the subject specification.
B6 Mouse strain C57B1/65
Cre Cre recombinase
CSF Colony-stimulating factor
ES Embryonic stem
FSH Follicle-stimulation hormone
G-CSF Granulocyte Colony-stimulating factor
GM-CSF Granulocyte-Macrophage Colony stimulating factor
LH Lutenising hormone
M-CSF Macrophage Colony-stimulating factor
neo^{r} Neomycin phosphotransferase gene conferring resistance to neomycin
PBS Phosphate buffered saline
PGK Phosphoglycerate kinase
SDS-PAGE Sodium diodecyl sulphate
tk Thymidine kinase
TUNEL Terminal transferase-mediated dUTP nick-end labelling
wk Week
wt Wild type.

### DETAILED DESCRIPTION OF THE PREFERRED EMBODIMENTS

The present invention provides a modified animal or avian species exhibiting reduced levels of a Bcl-w protein and/or a protein associated with Bcl-w or a derivative or homologue thereof, wherein said animal or avian species has an incapacity or a reduced capacity to induce or facilitate spermatogenesis.

Reference herein to a “Bcl-w” protein includes reference to a protein having an amino acid sequence substantially as set forth in SEQ ID NO:2 or SEQ ID NO:4 or an animo acid sequence having approximately 47% or greater similarity to either of SEQ ID NO:2 or SEQ ID NO:4. The nucleotide sequence set forth in SEQ ID NO:1 represents the human bcl-w gene while SEQ ID NO:3 is the murine bcl-w gene. The present invention extends, therefore, to Bcl-w with an amino acid sequence substantially as set forth in SEQ ID NO:2 or SEQ ID NO:4 as well as homologues, analogues or derivatives having at least about 47% similarity to the amino acid sequence set forth in SEQ ID NO:2 or SEQ ID NO:4. The Bcl-w protein or its homologues or derivatives are encoded by a nucleotide sequence substantially as set forth in SEQ ID NO:1 (human) or SEQ ID NO:3 (murine) or a nucleotide sequence having at least 47% similarity thereto and/or which is capable of hybridising thereto under low stringency conditions at 42° C. All such derivatives and homologues are encompassed by the terms “Bcl-w” (for the protein) or “bcl-w” (for the nucleic acid). Examples of derivatives of bcl-w include the nucleotide sequence set forth in SEQ ID NO:5 (human) or SEQ ID NO:7 (murine) or their corresponding amino acid sequences (SEQ ID NO:6 and SEQ ID NO:8, respectively). Wild type bcl-w may also be defined by reference to a nucleotide sequence capable of hybridising to a derivative of SEQ ID NO:1 or SEQ ID NO:3, such as SEQ ID NO:5 or SEQ ID NO:7.

Accordingly, another aspect of the present invention provides a modified animal or avian species exhibiting reduced levels of a Bcl-w protein having an amino acid sequence substantially as set forth in SEQ ID NO:2 or SEQ ID NO:4 or a Bcl-w protein encoded by a nucleotide sequence substantially set forth in SEQ ID NO:1 or SEQ ID NO:3 or a nucleotide sequence capable of hybridising to SEQ ID NO:1 or 3 or 5 or 7 under low stringency conditions at 42° C. wherein said animal or avian species has an incapacity or a reduced capacity to induce or facilitate spermatogenesis.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity. Any number of programs are available to compare nucleotide and amino acid sequences. Preferred programs have regard to an appropriate alignment. One such program is Gap which considers all possible alignment and gap positions and creates an alignment with the largest number of matched bases and the fewest gaps. Gap uses the alignment method of Needleman and Wunsch (20). Gap reads a scoring matrix that contains values for every possible GCG symbol match. GAP is available on ANGIS (Australian National Genomic Information Service) at website http://mel1.angis.org.au..

Reference herein to a low stringency at 42° C. includes and encompasses from at least about 1% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01M to at least about 0.15M salt for washing conditions.

Preferably, the percentage similarity or identity at the amino acid or nucleotide levels is between 48% and 100% inclusive such as approximately 50% or 55%, 59% or 65%, 70% or 75%, 80% or 85%, 90% or 95% or greater than 96% or a percentage similarity or identity there between.

A gene associated with bcl-w or a protein associated with Bcl-w includes the gene which is approximately 9.2 kb down stream of bcl-w exon 3 and which has homology to the Drosophila rox gene (13). Fusion RNA transcripts have been observed between bcl-w and rox and, hence, disruption of the rox gene or its transcript or translation production may impact on bcl-w expression or Bcl-w activity. The present invention extends, therefore, to targeting Rox, rox, bcl-w-rox fusion transcripts and Bcl-w-Rox fusion translation products. The present invention extends to other genes associate with bcl-w at the regulation, transcription or proximity levels.

Preferably, the Bcl-w protein is of mammalian origin such as from humans, primates, livestock animals (eg. sheep, cows, horses, pigs), companion animals (eg. cats, dogs), laboratory test animals (eg. rabbits, mice, rats, guinea pigs) and captive wild animals (eg. foxes, deer, kangaroos). However, the present invention also extends to non-mammalian homologues of Bcl-w such as from avian species, fish and reptiles. Generally, when producing a modified animal, the effector molecules to reduce Bcl-w activity or expression are identified on the basis of a Bcl-w from the same species. However, an effector molecule against, for example, murine Bcl-w may also be used against human Bcl-w. Both types of effector molecules are contemplated by the present invention and are referred to as heterologous or homologous effector molecules. Similar comments apply with respect to a gene associated with bcl-w or a protein associated with Bcl-w.

According to a particularly preferred embodiment, there is provided a modified animal exhibiting reduced levels of Bcl-w or a derivative or homologue thereof and/or of a protein associated with Bcl-w wherein said Bcl-w or its derivative or homologue comprises an amino acid sequence substantially as set forth in SEQ ID NO:2 or SEQ ID NO:4 or an amino acid sequence having at least about 47% similarity to the amino acid sequence of SEQ ID NO:2 or SEQ ID NO:4 and wherein said modified animal has a incapacity or a reduced capacity to induce or facilitate productive spermatogenesis.

In a related embodiment, there is provided a modified animal exhibiting reduced levels of Bcl-w or a derivative or homologue thereof and/or of a protein associated with Bcl-w wherein said Bcl-w or its derivative or homologue is encoded by a nucleotide sequence substantially as set forth in SEQ ID NO:1 or SEQ ID NO:3 or a nucleotide sequence having at least 47% similarity thereto and/or which can hybridise to SEQ ID NO:1 or SEQ ID NO:3 under low stringency conditions at 42° C.

The “modified” animal may be modified at the level of Bcl-w family protein activity or at the genetic level of the bcl-w gene. In regards to the former, the present invention contemplates the administration of a range of antagonists to Bcl-w protein activity resulting in reduced or substantially total removal of Bcl-w protein activity. For example, a vaccine may be administered containing Bcl-w protein or an immunogenic derivative thereof to induce antibodies to endogenous Bcl-w protein. Alternatively, a molecule identified from natural product screening capable of acting as an antagonist may be employed. Due to the intracellular nature of Bcl-w, antagonists are generally small molecules or in a form capable of entry into cells. A particularly important potential antagonist is a molecule containing a BH3 amino acid motif. The term “BH” stems from “Bcl-2 Homology” and relates to regions of homology between Bcl-2 proteins (reviewed by Kroemer (8)). The BH3 domain is capable of binding to Bcl-2 and related molecules. Accordingly, a small molecule, for example, a peptide comprising a BH3 motif or closely related to it, or a chemical mimetic thereof may provide antagonist activity towards Bcl-w. Similar considerations apply in respect of a gene or protein associated with bcl-w or Bcl-w, respectively.

The present invention further contemplates the use of naturally occurring molecules such as Bim (37) to regulate Bcl-w activity. Such molecules interact or otherwise associate with Bcl-w activity. Such molecules interact or otherwise associate with Bcl-w to modulate its activity.

The present invention further contemplates genetic vaccinations. For example, a DNA vaccine may be prepared in order to induce an immune response against Bcl-w. Enhanced immunogenicity may be obtained using molecular adjuvants such as a peptide derived from the C3d region which binds to the CR2 receptors on B cells (21). Other suitable molecule adjuvants include L. selectin and cytotoxic T-lymphocyte anigen (CTLA4) (22) or CD40 (23).

According to another aspect of the present invention there is provided a modified animal exhibiting an incapacity or a reduced capacity to induce or facilitate productive spermatogenesis said modification comprising the administration to said animal of an antagonistic effective amount of a molecule capable directly or indirectly of antagonising Bcl-w protein activity or the ability of a derivative or homologue of Bcl-w.

Examples of molecules directly affecting Bcl-w protein activity include an antibody, a soluble receptor for Bcl-w protein and a chemical found from natural product screening or the screening of synthetic libraries. An example of a molecule indirectly affect Bcl-w family protein activity includes a Bcl-w protein or an immunogenic derivative thereof capable of inducing an immune response against an endogenous Bcl-w protein. Another example is a molecule which targets a gene or protein associated with bcl-w/Bcl-w. As stated above, these molecules may need to be modified to permit entry into target cells.

In a related embodiment, there is provided a composition capable of inducing infertility or reducing fertility in an animal, said composition comprising a direct or indirect antagonist of a Bcl-w protein.

Reference to “natural product screening” includes products identified from sources such as but not limited to coral, soil, seabeds and sea water, bacteria, yeasts, plants and river water and river beds.

The composition of this aspect of the present invention may also comprise one or more carriers and/or diluents. Preferably the carriers are pharmaceutically acceptable.

The target animals are as stated above such as humans, primates, livestock animals, laboratory test animals and companion animals. The preferred modified animal, however, for the purposes of an in vivo model is a mouse, rat, rabbit, guinea pig, sheep or pig. The most preferred animal is a mouse.

Another aspect of the present invention relates to the genetic reduction in Bcl-w protein levels. According to this aspect of the present invention, there is provided a genetically modified animal comprising a mutation in one or more alleles of a gene encoding a Bcl-w protein and/or of a gene associated with Bcl-w protein.

In a related embodiment, there is provided a genetically modified animal comprising a mutation in one or more alleles of a gene comprising a sequence of nucleotides substantially as set forth in SEQ ID NO:1 or SEQ ID NO:3 or a nucleotide sequence having at least about 47% similarity thereto and/or a sequence which is capable of hybridising to SEQ ID NO:1 or SEQ ID NO:3 under low stringency conditions at 42° C.

Preferably, in order to observe the infertility phenotype, the animal model comprises an animal with a mutation in both alleles of bcl-w and is referred to as “bcl-w^{Δ/Δ}” which is considered equivalent to the designation “bcl-w^{-/-}”. An animal with a mutation in one copy of the gene is referred to as “bcl-w^{+/Δ}” or “bcl-w^{+/-}”. A bcl-w^{+/Δ} animal is also useful as a carrier for the bcl-w^{Δ/Δ} genotype. Reference to a bcl-w^{Δ} genotype is not to imply deletion of the entire coding region for Bcl-w although such a deletion is contemplated by the present invention. Partial deletion or any nucleotide insertion, deletion and/or addition is encompassed by the term “bcl-w^{Δ/Δ}” or “bcl-w^{Δ/+}”.

In accordance with the present invention, animals and in particular mice carrying a mutation in the bcl-w gene have normal populations of lymphoid, myeloid and erythroid cells in bone marrow, spleen, thymus and peripheral blood and normal numbers of haematopoietic progenitors in bone marrow. Adult female bcl-w^{Δ/Δ} mice are fertile. However, adult male bcl-w^{Δ/Δ} mice are infertile and have small testes. There are no other major abnormalities as determined by, for example, histological examination. The bcl-w^{Δ/Δ} mice grow more slowly after puberty than wild-type littermates. The structure of the seminiferous tubules of adult bcl-w^{Δ/Δ} mice is disorganised and the tubules are difficult to categorise according to the normal spermatogenic cycle. Heterogeneous degeneration of all germ cell types is evident, with some degenerating giant cells visible in the tubule lumen. While some round spermatids are present, there are few metamorphosing spermatids and no mature sperm. Seminiferous tubules of bcl-w^{Δ/Δ} mice contain increased numbers of apoptotic nuclei which label with the TUNEL technique, compared to tubules of wild-type littermates. The testes of 2 week old and 4 week old bcl-w^{Δ/Δ} mice appear grossly normal and contain some metamorphosing spermatids.

The term “mutation” is used in its broadest sense and includes a single or multiple nucleotide substitution, deletion and/or addition to bcl-w or to a region controlling bcl-w expression such as a promoter, polyadenylation signal or regulatory gene. The mutation generally results in no active Bcl-w protein being produced or substantially reduced levels of Bcl-w protein being produced. The mutation may also involve a splice variant. The mutation may also be outside the bcl-w gene but in a gene associated with bcl-w such as the rox gene. The term bcl-w^{Δ/Δ} denotes the absence of a functional Bcl-w protein. For convenience, it is also used to cover reduced levels of functional Bcl-w such as in the case of the administration of an antagonist of Bcl-w or if antisense molecules are used to induce a transient reduction in Bcl-w levels.

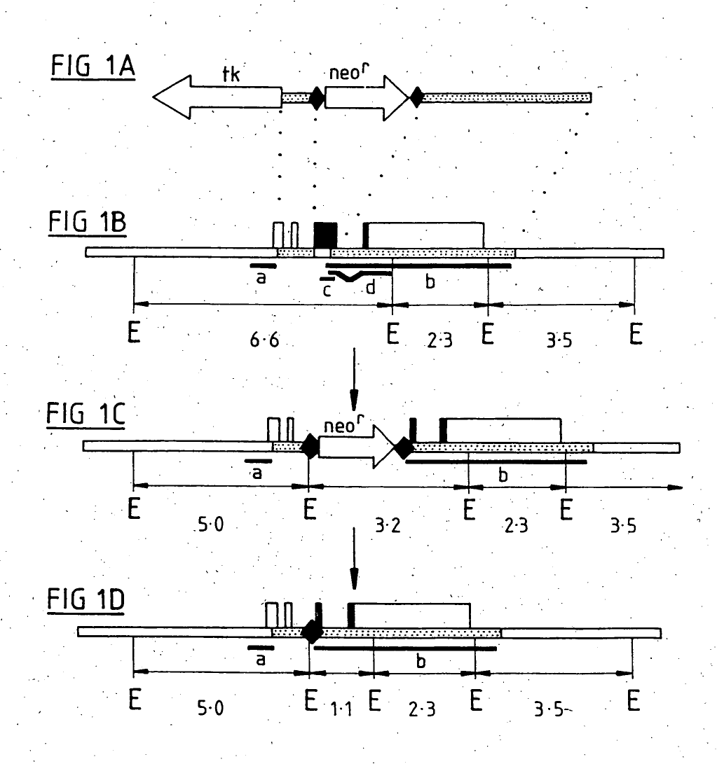

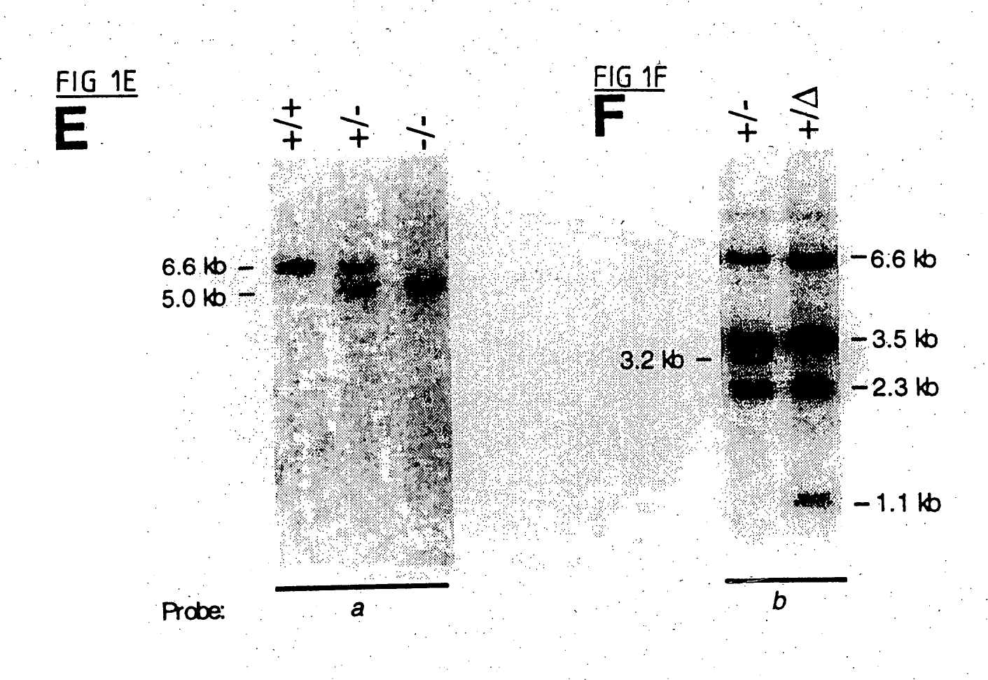

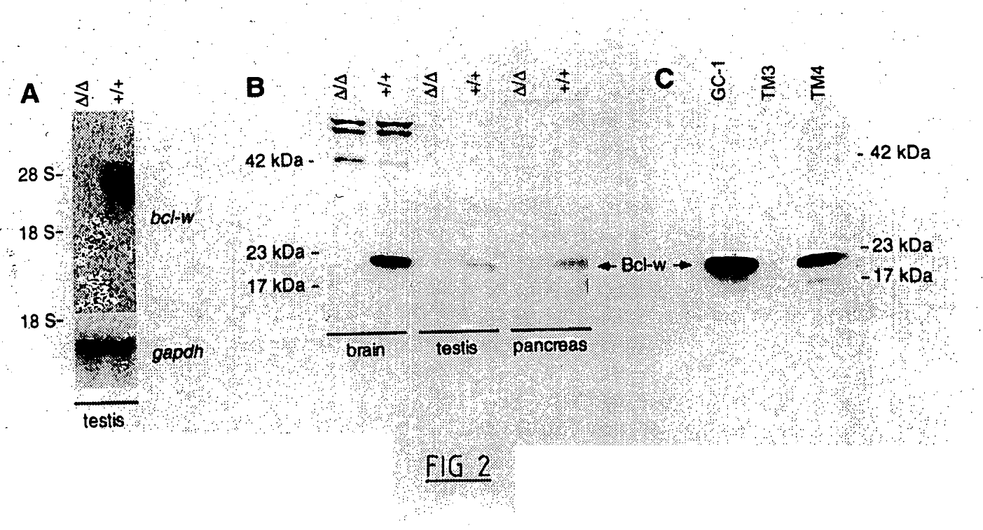

In a particularly preferred embodiment, a substantial portion of the gene has been deleted through, for example, homologous recombination. One particularly useful method is depicted in FIG. 1. According to this preferred method a plasmid targeting vector is prepared (eg. denoted lox-neo bcl-w) and transfected into embryonic stem (ES) cells. ES cell lines carrying one copy of the targeted bcl-w locus are generated and injected into blastocysts to produce chimeric mice. A targeting vector is preferably designed to replace almost the entire bcl-w coding sequence with a pgk-neo expression cassette. The pgk-neo cassette is bounded by sites (loxP) that allow its subsequent excision by the action of the bacteriophage Cre recombinase. In order to achieve this, chimeric mice carrying the bcl-w mutation have been bred with mice expressing a Cre transgene. The correct disruption of the bcl-w locus by homologous recombination and removal of the selectable marker by Cre-mediated recombination is confirmed by polymerase chain reaction and Southern blotting. Subsequent breeding generates bcl-w^{Δ/Δ} mice. A similar approach can be used to mutate a gene associated with bcl-w.

There are a number of other mechanisms for generating bcl-w^{Δ/Δ} mice or bcl-w^{+/Δ} mice and all these are encompassed by the present invention.

In addition, the present invention further contemplates transient disruption of the bcl-w gene through use of antisense molecules, ribozymes and deoxyribozymes. Viruses may also be employed to introduce antisense molecules or other molecules capable of disrupting function of the bcl-w gene. All such genetic molecules are encompassed by the present invention.

Another aspect of the present invention contemplates a method of producing a genetically modified animal substantially incapable of producing Bcl-w, said method comprising introducing a genetic sequence into ES cells, which genetic sequence targets the bcl-w gene or a gene associate with bcl-w and introducing said ES cells into blastocysts to produce chimeric mice.

The genetic sequence permits excision of the bcl-w gene or a selectable marker or specific region within or associated with the bcl-w gene by, for example, Cre recombinase.

Preferably, the animal is a mouse.

The ES cells may be from the recipient animal (allergenic) or from a different animal of the same species (heterogenic).

The modified animals of the present invention are particularly useful in screening for genetic or non-genetic molecules capable of restoring fertility. They are also useful as a model for studying the effects of infertility and in the rationale design of molecules capable of inducing infertility.

The bcl-w^{Δ/Δ} mutation may also be linked to a “reporter” gene, such as could be used to illustrate expression of bcl-w in adult male mice and/or in mouse embryos. For breeding and screening purposes, such a readily identifiable marker would greatly facilitate the identification of bcl-w^{Δ/Δ} mice.

Agonists and antagonists of bcl-w or Bcl-w are also readily obtained by screening for molecules capable of interacting with the protein or modifying bcl-w expression. One useful assay involves culturing cells which are bcl-w^{+/+} or bcl-w^{Δ/Δ} and adding potential modulators and screens for apoptosis or reversal of apoptosis.

A further embodiment of the present invention contemplates transgenic animals such as mice containing a genetic sequence operably linked to a testis-specific promoter, which genetic sequence is capable of disrupting the bcl-w gene or bcl-w gene expression or expression of a gene associated with bcl-w in the testis.

Yet a further embodiment of the present invention is directed to a modified animal comprising a mutation in a gene corresponding to bcl-w or a derivative or homologue thereof or in a gene associated with bcl-w wherein an adult male of said animal exhibits the following characteristics:
(i) is substantially infertile;
(ii) possesses disorganised seminiferous tubules;
(iii) exhibits heterogenous degeneration of germ cell types; and
(iv) possesses no other major abnormalities as determined by histological examination.

In murine and human species the bcl-w mutation is on chromosome 14 and specifically 14q11 in humans. It may be located on other chromosomes in other species.

Yet a further embodiment of the present invention contemplates an animal model for studying other degenerative disorders such as but not limited to neurodegenerative disorders. For example, animals such as mice which are bcl-w^{+/Δ} or bcl-w^{Δ/Δ} in glial cells may ultimately develop a neurodegenerative disorder. Such animal models would be useful in screening for genetic and therapeutic molecules capable of treating such degenerative disorders. Cell lines which are bcl-w^{+/+} or bcl-w^{Δ/Δ} are also contemplated to be useful in screening assays.

The present invention is further described by the following non-limiting Examples.

Examples 1 to 9 provide the materials and methods employed to obtain the data of Example 10.

### EXAMPLE 1

### Disruption of bcl-w

The bcl-w gene was inactivated by homologous recombination. The gene targeting vector (see FIG. 1A) was assembled in ploxPneo-1 in which a neomycin phosphotransferase gene (neo^{r}), driven by a phosphoglycerate kinase (PGK) promoter, is flanked by bacteriophage P1 loxP sites. The 129/Sv mouse bcl-w genomic DNA sequences introduced at each end of the loxP-neo^{r}-loxP cassette comprised the 876 bp region immediately upstream of the bcl-w start codon and the 4-kb Bam HI fragment extending from within exon 3 through the entire 3′ untranslated region. Introduction of a terminal herpes simplex virus thymidine kinase (tk) gene driven by a PGK promoter then completed the vector (FIG. 1A), which was linearized and electroporated into W9.5 ES cells (24). ES cell clones selected for resistance to G418 (i.e. neo^{r} gene integration) and gancyclovir (i.e. loss of the tk gene following homologous recombination) (25) were screened for homologous recombination at the bcl-w locus by Southern blot analysis. The bcl-w mutant ES cell clones were injected into the blastocoel cavity of C57BL/6J (B6) blastocysts, which were then implanted into pseudopregnant foster mothers. Male chimeric progeny were crossed to B6 females or, to delete the neo^{r} cassette, to B6/FVB F1 females expressing bacteriophage P1 Cre recombinase (Cre) (26).

### EXAMPLE 2

### Analysis of Mouse Weights

Wild type (wt) and mutant mice were weighed weekly from birth to 20 wk, and the weights analyzed using the split-line model (27). Briefly, growth curves before and after puberty were fitted to two straight lines, and the slopes of these lines and their point of intersection compared.

### EXAMPLE 3

### Blot Analysis

Southern blot analysis on cultured ES cells or mouse tail tips used 500-bp Stu I-Bam HI and 4-kb Pml I genomic DNA fragments (probes a and b respectively in FIG. 1B). Northern blot analysis was conducted on total RNA (10 μg/lane) prepared (28) from testes of adult mice. For western blot analysis, tissues or cells were washed in phosphate-buffered saline (PBS), immediately frozen in isopentane on dry ice, homogenized at 4° C. in buffer (50 mM TrisHCl (pH 7.5), 2 mM EDTA, 1 % Nonidet P-40) containing 1 mM phenylmethylsulfonyl fluoride, 2 μg/ml aprotinin, 1 μg/ml pepstatin and 2 μg/ml leupeptin and then centrifuged at 10,000×g at 4° C. for 30 min. Proteins (35 μg) in the supernatant were resolved by SDS-PAGE (12% w/v acrylamide gel) and transferred to nitrocellulose membranes (Hybond-C extra, Amersham). As controls for protein loading and integrity, membranes were stained with Ponceau S, or with an antibody against the ubiquitous Hsp-70. Bcl-w was detected by incubation of the membranes overnight with a polyclonal rabbit-anti-human Bcl-w antibody (AAP-050, StressGen Biotechnologies), followed by horseradish peroxidase-conjugated goat anti-rabbit antibody (Selenius) and chemiluminescent reagents (Amersham).

### EXAMPLE 4

### Histology and BrdUrd Labelling

Tissues fixed in Bouin's solution for 5 hr were embedded in paraffin, and 8 μm sections transferred to silane-coated microscope slides and stained with hematoxylin and eosin. The following tissues were examined: brain, colon, salivary gland, liver, heart, stomach, skeletal muscle, skin, peripheral nerve, pituitary gland, eye, teeth, bone, cartilage, thyroid and parathyroid glands, blood vessels, lung, small intestine, pancreas, kidney, adrenal gland, bladder, uterus, ovary and testis. To determine mitotic turnover, mice were injected i.p. with BrdUrd (100 μg/g body weight in 7 mM NaOH) 8 hr before sacrifice. Paraffin-embedded sections of testis, small intestine, colon, spleen, thymus and bone marrow were stained with rat-anti-BrdUrd antibody (Mas 250P, Harlan Ser-Lab). This was detected by biotinylated mouse-anti-rat Igκ antibody (Mar 18.5), avidin-biotinylated horseradish peroxidase (Elite ABC, Vector Laboratories) and diaminobenzidine.

### EXAMPLE 5

### Terminal Transferase-Mediated dUTP Nick End-Labelling (TUNEL)

Paraffin-embedded sections were treated with 20 μg/ml proteinase K in water for 15 min at room temperature, then DNA free ends were labelled with dUTP-biotin using terminal deoxynucleotidyl transferase (29) and revealed with avidin-biotinylated horseradish peroxidase. For each testis, TUNEL-labelled (apoptotic) nuclei in approximately twenty-five 0.56 mm² fields were counted, and the number of apoptotic nuclei per seminiferous tubule determined.

### EXAMPLE 6

### Hematologic Analysis

Peripheral blood erythrocytes and leucocytes were enumerated using a Coulter counter, and platelets with a Sysmex NE8000 counter (TOA, Kobe, Japan). Leucocytes in peripheral blood, femoral bone marrow, peritoneum, spleen and thymus were stained with eosin and counted by hemocytometer. Cytocentrifuge preparations were stained with May-Grunwald-Giemsa. Single cell suspensions prepared from blood, bone marrow, spleen and thymus were incubated with 2.4G2 anti-Fcγ receptor antibody (30) to reduce background staining, labeled with fluorescent surface marker-specific monoclonal antibodies and analysed by flow cytometry as elsewhere described (31).

To enumerate progenitor cells, bone marrow and spleen cells were cultured in medium containing 0.1% w/v agar (32) and the following cytokines: 10 ng/ml murine granulocyte-macrophage-colony stimulating factor (GM-CSF), 10 ng/ml human granulocyte-CSF (G-CSF, 10 ng/ml murine macrophage-CSF (M-CSF), 10 ng/ml murine interleukin-3, 100 ng/ml murine stem cell factor or 200 ng/ml murine thrombopoietin. To determine the cellular composition of each colony, the agar plates were fixed and stained for acetylcholinesterase, then with Luxol fast blue and hematoxylin (32).

### EXAMPLE 7

### Testis Stereology

Testes fixed for 5 hr in Bouin's fixative were embedded in methacrylate; 25 μm sections were transferred to glass slides and stained with hematoxylin and the periodic acid-Schiff reagent. Leydig and Sertoli cells and germ cells were counted using the ‘optical disector’ approach as described previously (33).

### EXAMPLE 8

### In Situ Hybridisation

Digoxigenin-labelled riboprobes were generated from linearized plasmid DNA templates (34). Riboprobes c1 (sense) and c2 (anti-sense) (FIG. 1B) were generated from residues 118 to 410 of the bcl-w cDNA (GenBank U59746) in the pT7Blue vector (Novagen), and d1 and d2 from residues 330 to 956 in the pBSIISK vector (Stratagene). Paraffin-embedded tissue sections on microscope slides were treated with 1 μg/ml proteinase K in buffered saline for 30 min at 37° C., hybridized to the riboprobes at 50° C. for 16 hr, and washed to 0.1×SSC at 50° C. (34). Slides were then exposed to an alkaline phosphatase-conjugated anti-digoxigenin antibody (Boehringer Mannheim), riboprobes detected with the nitroblue tetrazolium chloride/bromo-chloro-indolyl phosphate substrate, and the slides counterstained with hematoxylin.

### EXAMPLE 9

### Serum Gonadotrophin Assay

The concentration of follicle-stimulating hormone (FSH) and luteinizing hormone (LH) in serum was determined by a double-antibody radioimmunoassay using reagents for the measurement of rat FSH and LH (35). Their efficacy on the mouse hormones was confirmed. All samples were measured in the same assay with an intra-assay coefficient of variation of 4.8% and 5.9% for the FSH and LH assays, respectively.

### EXAMPLE 10

### Results

Disruption of bcl-w

The gene targeting vector was designed to inactivate bcl-w by replacing the first two thirds of its coding region with a PGK-neo^{r} expression cassette bounded by loxP sites (FIG. 1A-C). Any translation of the remainder should be precluded by a preceding stop codon. Homologous recombination was obtained in 8 of 352 selected ES cell clones. The structure of the mutant allele (bcl-w⁻) was confirmed by Southern blot analysis: bcl-w probe a detected 6.6-kb and 5.0-kb Eco RI fragments diagnostic for the wt and bcl-w⁻ alleles, respectively (e.g. FIG. 1E). A neo^{r} probe excluded the presence of any copies of the targeting vector integrated elsewhere in the genome. Two independent recombinant ES clones were used to generate chimeric mice, which were bred with B6 females to generate two lines of bcl-w-mutant mice (228 and 229), each of which was subsequently bred to homozygosity.

Regulatory sequences introduced by gene targeting can inadvertently alter the expression of neighbouring genes. Just 5.5 kb downstream of bcl-w is the gene encoding poly (A)-binding protein II (mPABII (36), homologue of rox (19)). To avoid altering the expression of this or other neighboring genes, the inventors also generated mice in which the introduced PGK-neo^{r} cassette was deleted by crossing both 228 and 229 mice with animals expressing Cre recombinase at the 2-cell stage of development (3) (FIG. 1D). Progeny carrying the deleted allele (bcl-w^{Δ}, FIG. 1D) were recognized by a diagnostic 1.1-kb Eco RI fragment (FIG. 1F), and the deletion was confirmed by sequencing a PCR product spanning the recombination site. Crosses with B6 mice then generated lines 228Δ and 229Δ. Northern blot analysis confirmed that expression of the mPABII gene was unaffected in 228Δ mice homozygous for the bcl-w^{Δ} allele. Importantly, homozygous mutants of all four lines (228, 229, 228Δ and 229Δ) proved to be indistinguishable.

Bcl-w is Dispensable for Development

As expected, the bcl-w^{Δ/Δ}-mice expressed neither bcl-w RNA nor protein. No RNA transcript was detected by a bcl-w cDNA probe in northern blots of RNA extracted from testis (FIG. 2A), and western blots with an anti-Bcl-w antibody revealed no Bcl-w protein in lysates from brain, testis or pancreas (FIG. 2B).

Lack of Bcl-w did not compromise survival of fetal or neonatal mice. The offspring of bcl-w^{+/Δ} intercrosses were born at normal Mendelian frequency: 25% bcl-w^{+/+}, 47% bcl-w^{+/Δ} and 28% bcl-w^{Δ/Δ}, and 57% of bcl-w^{Δ/Δ} offspring were male (n total=210). The bcl-w^{Δ/Δ} mice exhibited no significant abnormality in external appearance or behavior. The growth of bcl-w^{Δ/Δ} pups from birth to 5 wk of age was indistinguishable from that of their wt littermates. Although the average weights of male and female bcl-w^{Δ/Δ} mice at 5, 7, 9, 12, 16 and 20 wk of age were slightly less than that of their bcl-w^{+/+} and bcl-w^{+/Δ} littermates, the differences were not statistically significant. In addition, the growth curves of wt and bcl-w^{Δ/Δ} mice were indistinguishable when analyzed using the split-line method (27). Thorough histological examination of numerous tissues (see Examples 1 to 9) from bcl-w^{Δ/Δ} mice 6 and 52 wk of age revealed no significant abnormalities.

Normal Maintenance of Hematopoiesis

Since bcl-w RNA is detectable in most myeloid and some lymphoid cell lines (19), the hematopoietic tissues of bcl-w^{Δ/Δ} mice were carefully scrutinized. In mice analyzed at 6 and 52 wks, the weight and histology of the thymus, spleen, lymph node and bone marrow were normal. Blood cell analysis of three adult mice indicated normal numbers of erythrocytes, platelets, neutrophils, monocytes, eosinophils and lymphocytes (B and T). The peritoneal leucocyte population was also unaffected. The frequency of apoptotic nuclei in the spleen, thymus and bone marrow was unaltered, as judged by TUNEL analysis (29). Bcl-2 family members can slow mitotic cycle entry, but immunohistochemistry of spleen, thymus and bone marrow from bcl-w^{Δ/Δ} mice injected with BrdUrd 8 hours before sacrifice (see Examples 1-9) indicated normal numbers of leucocytes in the S phase.

Clonogenic assays on bone marrow cells from three adult bcl-w^{Δ/Δ} mice and three wt littermates yielded a comparable frequency of neutrophil, neutrophil-macrophage, macrophage, eosinophil, megakaryocyte and blast cell colony-forming cells, and the colonies were of similar size and maturation. Moreover, the progenitors were not rendered more sensitive to cytokine deprivation, since a 4-day delay in addition of interleukin-3 to such cultures reduced the number of colonies from wt and mutant marrow to equivalent extents.

Bcl-w is Essential for Spermatogenesis

Female bcl-w^{Δ/Δ} mice were fertile and competent to feed their pups. Intriguingly, however, all the males were infertile. While their external genitalia and testicular descent appeared normal, the cauda epididymides of bcl-w^{Δ/Δ} mice of all ages were devoid of sperm. In contrast, male heterozygotes exhibited normal fertility and epididymal histology.

Spermatogenesis involves an orderly process of germ cell maturation towards the center of the seminiferous tubules: mitotic proliferation of spermatogonia (up to 9 divisions), meiotic division of spermatocytes, differentiation of spermatids and finally release of spermatozoa into the tubule lumen. Histological examination of the testes of adult bcl-w^{Δ/Δ} mice revealed extensive albeit heterogeneous pathology within the seminiferous tubules. The tubules were abnormally small in diameter and often lacked a lumen. Numerous degenerating cells appeared throughout the seminiferous epithelium, some in the form of symplasts, giant cells containing several degenerating nuclei. There were few elongating spermatids more advanced than stage 13 of the seminiferous cycle and no mature sperm. Indeed, by 52 wk of age, almost no germ cells were discernible, although Sertoli cells remained. The defect was not in proliferation, since anti-BrdUrd-immunohistochemistry revealed numerous spermatocytes in S phase. Instead there was a striking elevation in the number of TUNEL-labelled apoptotic cells, many of which were contained within symplasts.

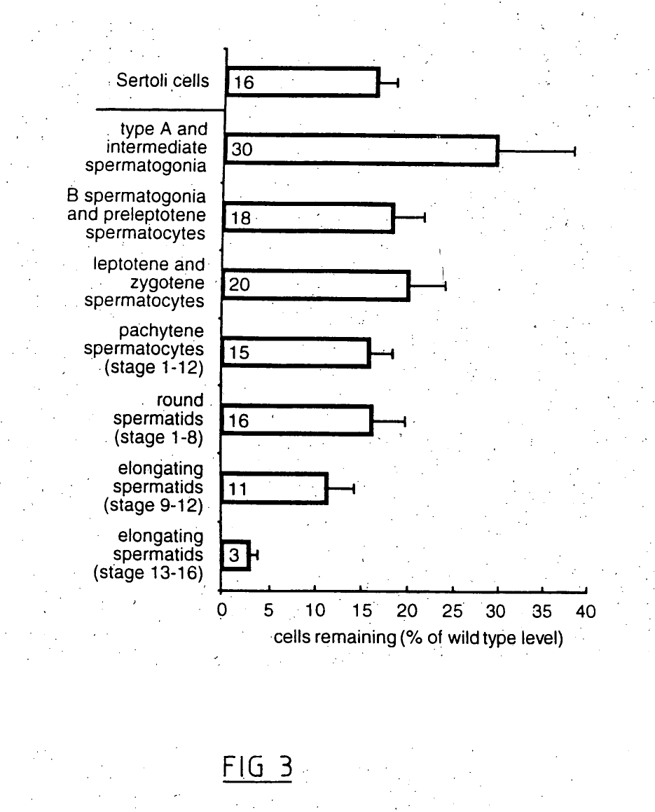

To determine which cells were affected, the inventors used the well-characterized ‘optical disector’ method (see Examples 1-9) to calculate the total number of each cell type within the testes of wt and bcl-w^{Δ/Δ} mice at 6 wk of age. Leydig cells were increased by nearly 50%. For each of the other cell types analyzed, however, mutant testes contained significantly fewer cells than wt testes (Student s t-tests, P<0.05). Sertoli cell numbers had decreased to 16% of their normal level (FIG. 3). Interestingly, germ cell numbers declined progressively with advancing stages of differentiation. Whereas type A spermatogonia were 30% of the normal level, spermatocytes represented only 15% to 20% of normal numbers, and, during spermatid differentiation, the level fell to 3% of normal (FIG. 3). Cells were also enumerated in the testes of single wt and bcl-w^{Δ/Δ} mice at 12, 14 and 16 wk of age. The deficit of round and elongating spermatids was more severe by 12 wk of age, and by 14 wk very few cells at or beyond the pachytene spermatocyte stage remained. Heterozygotes exhibited none of these alterations.

Germ Cell Apoptosis Increases Near Sexual Maturity

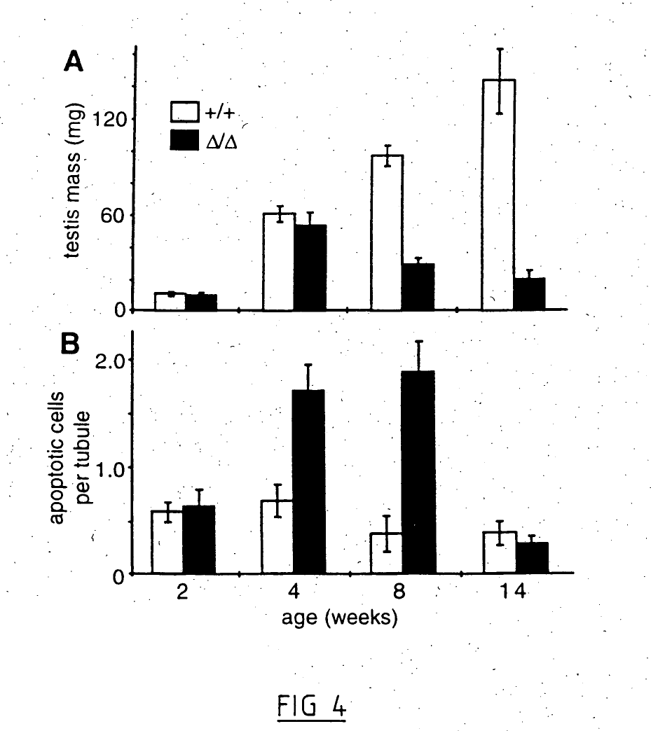

Early testicular development was normal. At 2wk of age, the testes of bcl-w^{Δ/Δ} mice exhibited normal mass and histology, and the number of TUNEL-labelled apoptotic nuclei per tubule was similar to that of wt littermates (FIG. 4). Even at 4 wk, the testes appeared normal and were of normal weight (FIG. 4A), suggesting that germ cell numbers had not yet fallen substantially, although there were twice as many apoptotic cells as in wt littermates (FIG. 4B). By 8 wk of age, however, the number of apoptotic cells was 5 times the normal level, and the testes had lost 70% of their mass (FIG. 4). Subsequently, the frequency of apoptotic cells declined, probably because so few germ cells remained. Thus, the apoptotic loss commences by 4 wk of age but severe attrition is evident only at sexual maturity.

No Evidence For an Endocrinological Basis

Germ cell apoptosis is inhibited directly by circulating androgens and FSH, and indirectly by LH, which promotes the secretion of androgens by leydig cells (32, 18). It seemed possible, therefore, that the spermatogenic defect was caused by reduced levels of these hormones. However, normal androgen levels could be inferred from the unaltered weight and histology of androgen-dependent organs (ventral prostate gland and seminal vesicles). Moreover, the serum FSH and LH concentrations of six wt and six bcl-w^{Δ/Δ} mice were equivalent (Student's t-test, P=1.0 for FSH and 0.1 for LH). These results, together with the normal histological appearance of the Leydig cells, hypothalamus and pituitary gland, make it unlikely that altered endocrine levels have a major role in the phenotype.

Expression of bcl-w in the Testis

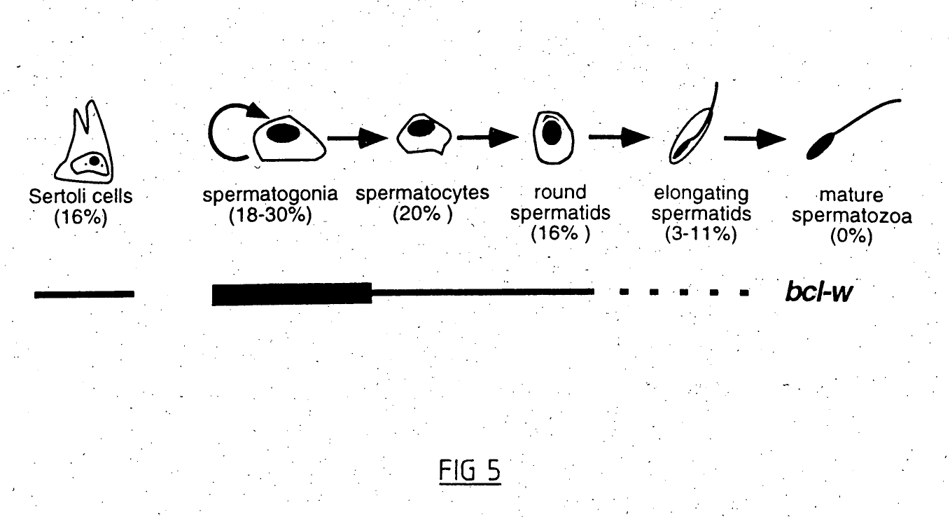

To facilitate interpretation of the phenotype of bcl-w^{Δ/Δ} mice, the inventors explored the expression pattern of bcl-w in wt adult testis. In situ hybridization indicated that bcl-w RNA was very prominent in the basal regions of seminiferous tubules. Antisense bcl-w riboprobes (c1 and d1, FIG. 1B) hybridized strongly to spermatogonia and moderately to spermatocytes, round spermatids and some Sertoli cells, but not detectably to elongating spermatids or mature sperm. Corresponding sense riboprobes (c2, d2) did not hybridize to any cell type and the antisense probes failed to detect any cells in the testis of bcl-w^{Δ/Δ} mice. Thus, bcl-w expression in adult testis was most conspicuous in pre-meiotic germ cells and was detectable in Sertoli cells but not in Leydig cells. The consequences of loss of Bcl-w in the testis is shown in FIG. 5.

The expression profile of Bcl-w in three mouse testicular cell lines was in accord with the in situ hybridization. Western blot analysis with a polyclonal anti-Bcl-w antibody revealed high levels of Bcl-w protein in the germ cell line GC-1 (derived from type B spermatogonia) and moderate levels in the Sertoli cell line TM4, but none in the Leydig line TM3 (FIG. 2C). Bcl-w was also detected in testes of 10-day old mice, which contain only Sertoli cells and spermatogonia.

Summary

Proteins of the Bcl-2 family are important regulators of apoptosis in many tissues of the embryo and adult. The recently isolated bcl-w gene encodes a novel pro-survival member of the Bcl-2 family which is widely expressed. To explore its physiological role, the inventors inactivated the bcl-w gene in the mouse by homologous recombination. Mice which lack Bcl-w were viable, healthy and normal in appearance. Most tissues exhibited typical histology, and hematopoiesis was unaffected, presumably due to redundant function with other pro-survival family members. While female reproductive function was normal, the males were infertile. The testes developed normally and the initial, prepubertal wave of spermatogenesis was largely unaffected. The seminiferous tubules of adult males, however, were disorganized, contained numerous apoptotic cells and produced no mature sperm. Both Sertoli cells and germ cells of all types were reduced in number, the most mature germ cells being the most severely depleted. The bcl-w^{Δ/Δ} mouse provides a unique model of failed spermatogenesis in the adult which has relevance to aspects of human male sterility.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Bibliography:

1. Tsujimoto et al. (1985) Science 228: 1440-1443.
2. Kerr et al. (1972) Br. J. Cancer 26: 239-257.
3. Strasser et al. (1997) Biochim. Biophys. Acta:, F151-F178.
4. Adams & Cory (1998) Science 281:1322-1326
5. Chao & Korsmeyer (1998) Annu. Rev. Immunol. 16: 395-419.
6. Oltvai et al. (1993) Cell 74: 609-619.
7. Pan et al. (1998) J. Biol. Chem. 273: 5841-5845.
8. Chinnaiyan et al. (1997) Science 275: 1122-1126.
9. Susin et al. (1996) J. Exp. Med. 184: 1331-1341.
10. Yang et al. (1997) Science 275: 1129-1132.
11. Kluck et al. (1997) Science 275: 1132-1136.
12. Veis et al. (1993) Cell 75: 229-240.
13. Kamada et al. (1995) Cancer Res. 55: 354-359.
14. Nakayama et al. (1993) Science 261: 1884-1888.
15. Nakayama et al. (1994) Proc. Natl. Acad. Sci. USA 91: 3700-3704.
16. Amling et al. (1997) J. Cell Biol. 136: 205-213.
17. Michaelidis et al. (1996) Neuron 17: 75-89.
18. Motoyama et al. (1995) Science 267: 1506-1510.
19. Gibson et al. (1996) Oncogene 13: 665-675.
20. Needleman and Wunsch (1970) J. Mol. Biol. 48: 443-453.
21. Lou and Kohler (1998) Nature Biotechnology 16: 458-462.
22. Boyle et al. (1998) Nature 392: 408-411.
23. Lane et al. (1993) J. Exp. Med. 177: 1209-1213.
24. Koentgen et al. (1995) Genes Develop 9: 1965-1977.
25. Mansour et al. (1988) Nature 336: 348-352.
26. Lakso et al. (1996) Proc. Natl. Acad. Sci. USA 93: 5860-5865.
27. Hudson, (1966) J. Am. Stat. Assoc. 61: 1097-1129.
28. Chomczynski & Sacchi (1987) Anal. Biochem. 162: 156-159.
29. Gorczyca et al. (1993) Cancer Res. 53: 1945-1951.
30. Unkeless (1979) J. Exp. Med. 150: 580-596.
31. Strasser et al. (1991) Cell 67: 889-899.
32. Metcalf, D. (1984) The Hemopoietic Colony Stimulating Factors. (Elsevier Science Publications, Amsterdam).
33. Wreford (1995) Microsc. Res. Tech. 32:
34. Meinhardt et al. (1998) J. Reprod. Fertil. 112: 233-241.
35. Lee et al. (1975) J. Reprod Fertil. 42: 121-126.
36. Lee et al. (1998) Biochemica et Biophysica Acta 1395: 40-46.
37. O'Connor et al. (1998) EMBO J 17(2): 384-395.

1-20. (canceled)

21. A genetically modified mouse comprising a homozygous disruption in an endogenous Bcl-2 gene, wherein said disruption results in reduced levels of or no Bcl-w protein, wherein said Bcl-w protein comprises an amino acid sequence set forth in SEQ ID No: 4 and wherein said male mouse has an incapacity or a reduced capacity when compared to a non-genetically modified male mouse to undergo spermatogenesis.

22. A homozygous genetically modified male mouse according to claim 21, wherein the Bcl-w protein is encoded by a nucleotide sequence as set forth in SEQ ID NO: 3 or a nucleotide sequence that hybridizes to SEQ ID NO: 3 under high stringency conditions at 42° C.

23. A homozygous genetically modified male mouse according to claim 21 or 22 wherein the mouse comprises a deletion in the bcl-w gene.

24. A homozygous genetically modified male mouse according to claim 21 or 22 comprising a mutation in one or more alleles of a gene which comprises a sequence of nucleotides as set forth in SEQ ID NO: 3.

25. A method of producing a homozygous genetically modified male animal incapable of producing Bcl-w, said method comprising introducing a genetic sequence into embryonic stem (ES) cells, which genetic sequence targets the bcl-w gene or a transcript thereof or a gene associated with bcl-w and introducing said ES cells into blastocysts to produce a chimeric mice.

26. A method according to claim 25 wherein the introduced genetic sequence is an antisense molecule, encodes an antisense molecule, is a sense molecule, encodes a sense molecule or permits excision of the bcl-w gene or a region within the bcl-w gene.

27. A method according to claim 26 wherein the introduced genetic sequence is bounded by sites that permit excision of the region between said sites by the action of a Cre recombinase.

28. A homozygous genetically modified male mouse comprising a mutation in the bcl-w gene or a derivative thereof wherein said mouse exhibits the following characteristics:
(i) is substantially infertile;
(ii) possesses disorganized seminiferous tubules;
(iii) exhibits heterogenous degeneration of germ cell types; and
(iv) possesses no other major abnormalities as determined by histological examination.

29. A homozygous genetically modified male mouse exhibiting reduced levels of a Bcl-w protein having an amino acid sequence as set forth in SEQ ID NO: 4 or a Bcl-w protein encoded by a nucleotide sequence set forth in SEQ ID NO: 3 or a nucleotide sequence that hybridizes to SEQ ID NO: 3 under high stringency conditions at 42° C. wherein said male mouse has an incapacity or a reduced capacity to undergo spermatogenesis.


SEQUENCE LISTING (8 sequences)

SEQ ID NO: 1 - DNA, 581 bp, Homo sapiens
  CDS (1)..(579)
atggcgaccccagcctcggccccagacacacgggctctggtggcagactttgtaggttataagctgaggcagaagggttatgtctgtggagctggccccggggagggcccagcagctgacccgctgcaccaagccatgcgggcagctggagatgagttcgagacccgcttccggcgcaccttctctgatctggcggctcagctgcatgtgaccccaggctcagcccaacaacgcttcacccaggtctccgatgaactttttcaagggggccccaactggggccgccttgtagccttctttgtctttggggctgcactgtgtgctgagagtgtcaacaaggagatggaaccactggtgggacaagtgcaggagtggatggtggcctacctggagacgcggctggctgactggatccacagcagtgggggctgggcggagttcacagctctatacggggacggggccctggaggaggcgcggcgtctgcgggaggggaactgggcatcagtgaggacagtgctgacgggggccgtggcactgggggccctggtaactgtaggggccttttttgctagcaagtg

SEQ ID NO: 2 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQEWMVAYLETRLADWIHSSGGWAEFTALYGDGALEEARRLREGNWASVRTVLTGAVALGALVTVGAFFASK

SEQ ID NO: 3 - DNA, 581 bp, Mus musculus
  CDS (1)..(579)
atggcgaccccagcctcaaccccagacacacgggctctagtggctgactttgtaggctataagctgaggcagaagggttatgtctgtggagctggccctggggaaggcccagccgccgacccgctgcaccaagccatgcgggctgctggagacgagtttgagacccgtttccgccgcaccttctctgacctggccgctcagctacacgtgaccccaggctcagcccagcaacgcttcacccaggtttccgacgaacttttccaagggggccctaactggggccgtcttgtggcattctttgtctttggggctgccctgtgtgctgagagtgtcaacaaagaaatggagcctttggtgggacaagtgcaggattggatggtggcctacctggagacacgtctggctgactggatccacagcagtgggggctgggcggagttcacagctctatacggggacggggccctggaggaggcacggcgtctgcgggaggggaactgggcatcagtgaggacagtgctgacgggggccgtggcactgggggccctggtaactgtaggggccttttttgctagcaagtg

SEQ ID NO: 4 - protein, 193 aa, Mus musculus
MATPASTPDTRALVADFVGYKLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQDWMVAYLETRLADWIHSSGGWAEFTALYGDGALEEARRLREGNWASVRTVLTGAVALGALVTVGAFFASK

SEQ ID NO: 5 - DNA, 583 bp, Homo sapiens
  CDS (1)..(579)
atggcgaccccagcctcggccccagacacacgggctctggtggcagactttgtaggttataagctgaggcagaagggttatgtctgtggagctggccccggggagggcccagcagctgacccgctgcaccaagccatgcgggcagctggagatgagttcgagacccgcttccggcgcaccttctctgatctggcggctcagctgcatgtgaccccaggctcagcccagcaacgcttcacccaggtctccgacgaactttttcaagggggccccaactggggccgccttgtagccttctttctctttggggctgcactgtgtgctgagagtgtcaacaaggagatggaaccactggtgggacaagtgcaggagtggatggtggcctacctggagacgcggctggtcgactggatccacagcagtgggggctgggcggagttcacagctctatacggggacggggccctggaggaggcgcggcgtctgcgggaggggaactgggcatcagtgaggacagtgctgacgggggccgtggcactgggggccctggtaactgtaggggccttttttgctagcaagtgaa

SEQ ID NO: 6 - protein, 193 aa, Homo sapiens
MATPASAPDTRALVADFVGYKLRQKGYVCGAGPGEGPAADPLHQAMRAAGDEFETRFRRTFSDLAAQLHVTPGSAQQRFTQVSDELFQGGPNWGRLVAFFLFGAALCAESVNKEMEPLVGQVQEWMVAYLETRLVDWIHSSGGWAEFTALYGDGALEEARRLREGNWASVRTVLTGAVALGALVTVGAFFASK

SEQ ID NO: 7 - DNA, 581 bp, Mus musculus
  CDS (1)..(579)
atgccgaccccagcctcaaccccagacacacgcgctctagtggctgactttgtaggctataggctgaggcagaagggttatgtctgtggagctgggcctggggaaggcccagccgccgacccgctgcaccaagccatgcgggctgctggagacgagtttgagacccgtttccgccgcaccttctctgacctggccgctcagctacacgtgaccccaggctcagcccagcaacgcttcacccaggtttccgacgaacttttccaagggggccctaactggggccgtcttgtggcattctttgtctttggggctgccctgtgtgctgagagtgtcaacaaagaaatggagcctttggtgggacaagtccaggattggatcgtggcctacctggagacacgtctggctgactggatccacagcagtggcggctgggcggacttcacagctctatacggggacggggccctggaggacgcacggcgtctgcgggagggcaactgggcatcagtgagcacagtggtgacgggggccgtggcactgggggccctggtaactgtaggggccttttttgctagcaagtg

SEQ ID NO: 8 - protein, 193 aa, Mus musculus
MPTPASTPDTRALVADFVGYRLRQKGYVCGAGPGEGPAADPLHQAMRAAGDEFETRFRRTFSDLAAQLHVTPGSAQQRFTQVSDELFQGGPNWGRLVAFFVFGAALCAESVNKEMEPLVGQVQDWIVAYLETRLADWIHSSGGWADFTALYGDGALEDARRLREGNWASVSTVVTGAVALGALVTVGAFFASK